(19) United States
(12) Patent Application Publication
(10) Pub. No.: US 2021/0275562 A1
(43) Pub. Date: Sep. 9, 2021

(54) CANCER TREATMENT METHODS

(71) Applicant: Dennis W. Stacey, Novelty, OH (US)

(72) Inventor: Dennis W. Stacey, Novelty, OH (US)

(21) Appl. No.: 17/249,528

(22) Filed: Mar. 4, 2021

Related U.S. Application Data
(60) Provisional application No. 62/985,347, filed on Mar. 5, 2020.

(51) Int. Cl. A61K 31/7072; A61K 9/00; A61K 31/475

(52) U.S. Cl. CPC A61K 31/7072; A61K 31/475; A61K 9/0019; A61K 9/0053

(57) ABSTRACT

A cancer treatment method includes administering a first dose of a drug selectively targeting cells in a particular phase (e.g., S phase cells); and administering an additional dose of the drug after waiting a full normal cell cycle. The method takes advantage of the uniformity of cell cycle length in normal cells (e.g., normal small intestine cells) and the and the lack of this uniformity, or variation in cell cycle length of cancer cells.

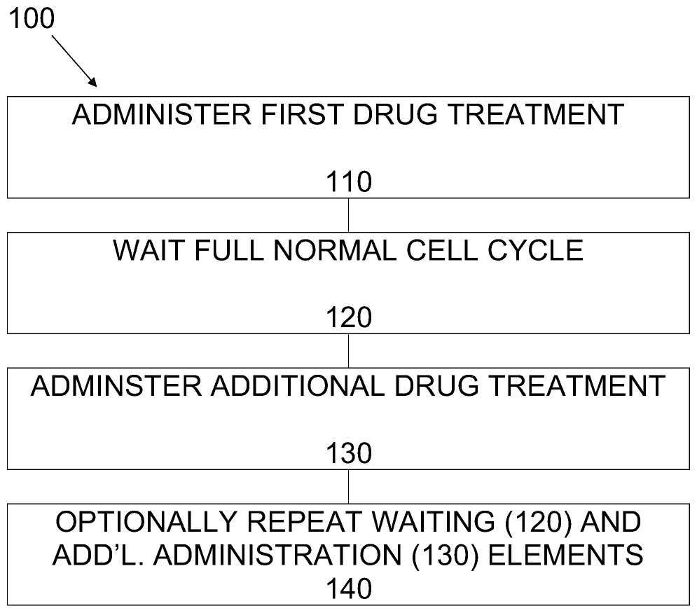

This application claims priority to U.S. Provisional Application Ser. No. 62/985,347, filed Mar. 5, 2020, which is incorporated by reference herein in its entirety.

### BACKGROUND

The present disclosure relates to cancer treatment methods that include the administration of cancer treatment drugs at particular times to take advantage of the cell cycle uniformity of some normal cells (e.g., small intestine cells) which is not possessed by many cancer cells.

Chemotherapy is a drug treatment that uses powerful chemicals to kill fast-growing cells (e.g., cancer cells).

Although chemotherapeutic drugs are effective for killing cancer cells, there are significant undesirable side effects, some of these and other effects may be attributed to the susceptibility of normal, healthy cells to the toxicity of chemotherapeutic drugs.

It would be desirable to develop new cancer treatment methods that more selectively target cancer cells while reducing damage to normal cells.

### BRIEF DESCRIPTION

Disclosed, in some embodiments, is a cancer treatment method including administering a first dose of a drug selectively targeting S phase cells; and administering an additional dose of the drug after waiting a full normal cell cycle.

Optionally, the method further includes repeating the administration of the additional dose at least one time.

The full normal cell cycle may be a normal human intestine cell cycle.

Alternatively, the normal cell cycle may be determined by the cell cycle length of cycling lymphocytes in the spleen; or bone marrow; or a combination of these.

The drug to be utilized must be specifically toxic to S phase cells and may be rapidly removed from circulation. It may be a nucleotide analogue, or an unrelated compound.

The means of identifying a suitable drug with the use of BRDU and EDU labelling, or a similar approach, is an aspect of this disclosure.

In some embodiments, the drug selectively targeting S phase cells is selected from the bromodeoxyuridine (BRDU) and 5-ethynyl-2′-deoxyuridine (EDU) family of molecules.

The drug selectively targeting S phase cells may be related to a dideoxynucleoside.

In some embodiments, the drug selectively targeting S phase cells is selected from the group consisting of methotrexate, pemetrexed, pralatrexate, cytarabine, gemcitabine, clofarabine, fludarabine, mercaptopurine, nelarabine, pentostatin, thioguanine, azacytidine, decitabine, irinotecan, and teniposide. These are drugs used in other treatment regiments but may also be appropriate in this one.

The drug selectively targeting S phase cells may be administered orally or via injection.

Disclosed, in other embodiments, is a process for treating small intestine cancer comprising in sequence: administering a first dose of a drug selectively targeting S phase cells, wherein the drug kills small intestine cancer cells in the S phase and normal small intestine cells in the S phase; and wherein the drug does not kill cells that are not in the S phase; waiting a full normal small intestine cell cycle; and administering an additional dose of the drug. This procedure is not specific to tumors of the gut but applies to any tumor.

Disclosed, in further embodiments, is a method of identifying an appropriate drug suitable for use in the treatment protocols described herein.

Disclosed, in additional embodiments, is a method of identifying appropriate drugs in a cell culture using BRDU and EDU markers. The results of this protocol may be predicted with the use of tritiated thymidine or another specifically S phase toxic drug in culture.

Also disclosed is a method to test potentially useful drugs in animals using BRDU and EDU staining methods.

Disclosed, in other embodiments, is a method to test a potentially useful drug and associated treatment protocol in animals containing or about to produce tumors; as a means to determine its antitumor potential.

### BRIEF DESCRIPTION OF THE DRAWINGS

The following is a brief description of the drawing, which is presented for the purposes of illustrating the exemplary embodiments disclosed herein and not for the purposes of limiting the same.

The FIGURE is a flow chart illustrating a non-limiting embodiment of a cancer treatment method in accordance with some embodiments of the present disclosure.

### DETAILED DESCRIPTION

The present disclosure may be understood more readily by reference to the following detailed description of desired embodiments included therein and the drawings. In the following specification and the claims which follow, reference will be made to a number of terms which shall be defined to have the following meanings.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent can be used in practice or testing of the present disclosure. All patent applications, patents, and other references mentioned herein are incorporated by reference in their entirety. The materials, methods, and articles disclosed herein are illustrative only and not intended to be limiting.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases that require the presence of the named ingredients/steps and permit the presence of other ingredients/steps.

However, such description should be construed as also describing compositions, mixtures, or processes as “consisting of” and “consisting essentially of” the enumerated ingredients/steps, which allows the presence of only the named ingredients/steps, along with any impurities that might result therefrom, and excludes other ingredients/steps.

Unless indicated to the contrary, the numerical values in the specification should be understood to include numerical values which are the same when reduced to the same number of significant FIGURES and numerical values which differ from the stated value by less than the experimental error of the conventional measurement technique of the type used to determine the particular value.

All ranges disclosed herein are inclusive of the recited endpoint and independently combinable (for example, the range of “from 2 to 10” is inclusive of the endpoints, 2 and 10, and all the intermediate values). The endpoints of the ranges and any values disclosed herein are not limited to the precise range or value; they are sufficiently imprecise to include values approximating these ranges and/or values.

As used herein, approximating language may be applied to modify any quantitative representation that may vary without resulting in a change in the basic function to which it is related. Accordingly, a value modified by a term or terms, such as “about” and “substantially,” may not be limited to the precise value specified, in some cases. The modifier “about” should also be considered as disclosing the range defined by the absolute values of the two endpoints.₁ For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Eukaryotic cells pass through a cell cycle including two gap phases (G1 and G2), a synthesis phase (S) during which genetic material is duplicated, and an M phase in which nuclear division (mitosis) is followed by cell division (cytokinesis). The phases in sequence are G₁→S→G₂→M. During the G₁ phase, the cellular content except for the chromosomes is duplicated. Next in the S phase, the chromosomes are duplicated by the cell. Subsequently in the G2 phase, the cell checks the duplicated chromosomes for errors and makes any necessary repairs. Nuclear and cell division occur in the M phase.

The S phase is of particular interest in accordance with the methods of the present disclosure. Accurate duplication of the genetic material is critical to cell division. The methods of the present disclosure take advantage of the degree of uniformity in cell cycle length (in some cases referred to as synchrony) of some normal cells (e.g., small intestine cells) by specifically targeting S phase cells in multiple treatment steps as discussed in more detail below.

In alternative embodiments, M phase cells may be similarly targeted using a drug that selectively kills mitotic cells (e.g., vinblastine and its derivatives). The M phase-targeting drug may block the formation of the mitotic spindle. It may bind tubulin and block the completion of mitosis.

The FIGURE is a flow chart illustrating a non-limiting embodiment of a cancer treatment method 100 in accordance with some embodiments of the present disclosure. The method includes administering a first drug treatment dose 110, waiting a full normal cell cycle 120, and administering an additional drug treatment dose 130. The waiting 120 and additional administering 130 are optionally repeated 140 one or more times.

The first treatment step is expected to kill both cancerous and normal cells but only those cells that are actively synthesizing DNA (i.e., in the S phase of the cell cycle). Cells in other phases should not be harmed because the treatment drug(s) specifically target S phase cells. In some embodiments, the first treatment step is applied when a high proportion of the normal or the cancer cells are in the S phase.

The second treatment step is similar to the first treatment step but applied only after the normal cells have progressed through the entire cell cycle. Waiting one full cycle can lead to the normal cells being in the same phase in both treatment steps. Because of this timing, a high proportion of the normal cells that were not making DNA at the time of the first treatment step will also not be making DNA at the time of the second treatment step. These cells would thus be protected from the second, and any subsequent treatment step(s).

The normal cells may be a population with highly similar cell cycle lengths, while the tumor cells presumably do not display the same degree of cell cycle uniformity, and are therefore, not protected. Extensive testing indicates tumors often have dramatically different average cell cycle lengths in comparison to normal cells. It is the difference in cell cycle length, thus identified, that is the basis of selectively killing the tumor cells. Emphasis has been made upon gut cells and lymphocytes because it is these tissues that are often particularly damaged in the course of chemotherapeutic treatments. Where other cell types may be limiting, these would also be considered. Thus, their normal cell cycle lengths would be determined and utilized as appropriate in accordance with the descriptions contained below.

Optionally, additional treatment steps may be applied. In general, each further treatment step may be applied after the cells have progressed through another complete cell cycle such that the protected normal cells from the first and second treatment steps will again be protected in the third, fourth, etc. treatment steps (i.e., they will be in the same phase in each treatment step). The number of further treatment steps may be one, two, three, four, five, six, seven, eight, nine, ten, or more.

The methods may be performed in vitro or in vivo. The methods may be performed on an animal, typically a mammal and more particularly a human. However, it is contemplated that the methods may also be performed on pets (e.g., cats and dogs) or other animals.

The inventor recently found that the length of the cell cycle of naturally growing cells in the mouse small intestine is short (approximately 14 hours) and displays limited variation between cells. In other words, most of the normal cells grow rapidly and with remarkably similar speeds. It is believed that human small intestine cells also exhibit uniform cell cycle lengths. Lymphocytes obtained from the spleen also exhibit a high degree of uniformity in cell cycle length. In the case of spleen cells, however, the cell cycle length is closer to 12 hours. A treatment protocol may utilize the length of the cell cycle in spleen or in gut, or an average between them. As indicated above, extensive studies of naturally occurring gut tumors in MIN mice have shown that many tumors have a dramatically different cell cycle timing than adjacent normal cells. Furthermore, the departure of tumors from the typical constraints observed in normal cells would suggest that in general, tumors would not display the same cell cycle lengths of normal cells. Finally, in most tumors, particularly more advanced ones, tumor cells do not behave uniformly as in normal tissues. Rather, tumor cells exhibit a wide range of growth and morphological characteristics. Tumor cells would, therefore, be expected to exhibit widely varied cell cycle times in general. In the studies conducted so far on naturally occurring gut tumors, these predictions have been conformed.

The treatment methods of the present disclosure are not limited specifically to small intestine cells or lymphocytes. Instead, they are believed to be applicable to any cell population with similar uniformity of cell cycle length. More extensive studies will undoubtedly refine the length of normal cell cycles in both tissues but are expected to be very near the numbers listed above.

All growing cells are located in one of the four cell cycle stages. Growing cells are generally randomly distributed among the different phases. Because these cells grow at similar rates, however, it is possible to predict when a given cell will return to a specific cell cycle stage. If the tumor treatment were designed to be non-toxic at a given cell cycle stage, the normal but not the tumor cells would be protected by virtue of the fact that the timing during which the normal but not the tumor cell will reside in the non-toxic cell cycle phase can be predicted. Cells synthesizing DNA, in S phase, are uniquely sensitive to inhibitors of DNA synthesis, or toxins that can be incorporated directly into DNA. Approximately half of proliferating cells in the crypt area of the gut are in S phase at any given time (further out in the villi the mature cells stop dividing altogether and these cells far from the basement membrane are not believed to be impacted by the methods of the present disclosure). If an animal were treated with a toxin for S phase cells, half the normal cells in this area would be killed, as would a large number of the growing tumor cells. There is no particular advantage to this approach for the first treatment. However, if one treatment was given, followed by a period equal or closely related to the length of the cell cycle before a second treatment was given, the normal cells that had survived the first treatment would largely be unsusceptible to the second. Cells that were not in S phase for the first treatment would once again not be in S phase for the second, and would, therefore, be protected from both. This approach could theoretically be repeated numerous times, with limited additional toxicity to normal cells. Because of different cell cycle times, however, the tumor cells would not be protected from the second or subsequent treatments.

The treatments do not need to be separated precisely by the determined cell cycle length of any particular normal cell type. Variations in the time between treatments are also envisioned. For example, if two normal tissues have similar cell cycle lengths, treatments may be spaced between them. Moreover, in the case that a tumor might have a cell cycle length slightly longer than that of a normal cell type, the treatments may be shorter than the calculated normal cell cycle length; so as to be more protective to the normal cells compared to the tumor. Moreover, the length of S phase is up to 6 hr in the cells analyzed. It is possible that treatment times equal to the cell cycle length plus or minus the length of S phase, or part of the length of S phase might be utilized. It is also possible that variations in the length of the cell cycle of normal cells might be considered. Even though the length of the cell cycle of normal crypt and spleen cells are relatively uniform, they are not of course perfectly uniform. The time between treatments with anti-cancer drugs described here might reflect not only the average cell cycle length, but the variations seen within the pool of normal cells. Finally, other, unforeseen circumstances might arise based upon the individual circumstances related to the cell cycle length of normal and tumor cells; necessitating an alteration in the time between drug treatments that are not specifically determined by the cell cycle length of either cell type. Such alternations as well as dosages in various steps are considered to be within the level of skill in the art familiar with the present application. The treatments are selected with a knowledge of the length of the cell cycle in normal cells compared to tumor cells; and which are most beneficial in tumor killing.

Although the cell cycle used to determine treatment time described above is related to gut or spleen cells, it should be understood that the treatment methods of the present disclosure are not necessarily specifically designed for gut or spleen tumors. Instead, the treatment methods are intended to protect normal gut cells in the treatment of tumors anywhere in the body. No matter where the tumor is located, a serious side effect of conventional chemotherapy treatments is toxicity to the gut and immune system. The treatment methods of the present disclosure are intended to reduce or eliminate this side effect. Very few, if any, tumors are believed to have the same cell cycle characteristics of normal gut cells.

It should also be understood that the cell cycle used to determine treatment time is not necessarily related to gut cells. However, the optimal length of time between treatments in the above approach may not always be identical to the cell cycle length. This may be necessitated by the fact that the S phase of growing cells is from 5-7 hr in length. The optimal timing of antitumor treatments may take the length of S phase into consideration, along with the overall length of the cell cycle of sensitive normal cells. The optimal cell cycle length may be for lymphoid cells. Spleen tissue itself does not proliferate. However, lymphoid cells residing in the spleen do proliferate. These lymphoid cells in the spleen exhibit a similar degree of cell cycle length uniformity compared to gut cells according to initial testing. Bone marrow contains a wide variety of proliferating cells. This tissue is particularly sensitive to many chemotherapeutic approaches. It may prove useful to identify the normal cell cycle length of a particularly sensitive or critical cell type in the bone marrow and base the treatment, at least in part, upon the cell cycle length of this cell type.

Sensitivity of lymphoid and gut cells are the primary limitations to many types of chemotherapy. In some embodiments, the methods of the present disclosure utilize timing intended to protect both of these cell types.

In other embodiments, the methods are timed based on the cell cycle of bone marrow cells, or specific cell types within the bone marrow, as an alternative to or in addition to one or both of the cell types discussed above.

The value is unique in that it is based upon a fundamental difference in the basic way normal and tumor cells grow. No known treatment strategy is believed to take advantage of this difference.

Non-limiting examples of S phase targeting drugs include bromodeoxyuridine (BRDU), 5-ethynyl-2′-deoxyuridine (EDU), deoxynucleosides, didanosine, zalcitabine, reverse transcriptase inhibitors, methotrexate, pemetrexed, pralatrexate, cytarabine, gemcitabine, clofarabine, fludarabine, mercaptopurine, nelarabine, pentostatin, thioguanine, azacytidine, decitabine, irinotecan, and teniposide.

BRDU is a DNA proliferation marker. It is incorporated into DNA. BRDU may be injected in a sterile solution or taken orally (e.g., in water or another beverage or in pill form).

EDU serves as a marker of DNA synthesis in individual cells and is incorporated into DNA like BRDU. EDU may be administered in a similar manner to BRDU.

One set of drugs to be tested are the dideoxynucleosides, that are capable of being incorporated directly into newly synthesized DNA. Once incorporated they would of necessity be S-phase specific in toxicity due to the fact that the block chain elongation of DNA. They are also believed to be rapidly removed from the blood stream as BRDU and EDU are. These may well be drugs currently utilized in other chemotherapeutic approaches, or drugs discarded in previous studies. However, drugs of this type currently utilized in treatment of Human Immunodeficiency Virus have been found to have reduced overall toxicity to mice, presumable due to their exclusion form the normal DNA synthetic machinery. For this reason, dideoxynucleosides that would be suitable for use in this protocol will need to be utilized with high efficiency by the normal DNA replicative machinery.

One non-limiting possibility relates to a traditional anti-cancer drug whose effects are still being studied, 5-fluorouracil (5-FU). It is the lead compound in a family of anticancer drugs, including tegafur, capecitabine: 5-fluorouridine 5′-triphosphate (FUTP), 5-fluoro-2′-deoxyuridine 5′-triphosphate (FdUTP), and 5-fluoro-2′-deoxyuridine 5′-monophosphate (FdUMP). While these are primarily known as thymidine synthase inhibitors, the latter is known to be incorporated into DNA and cause DNA damage and cell death. It, in particular, may be suitable for use in the above strategy. Tegafur and capecitabine are prodrugs for 5-FU that are readily absorbed in the gut. This family of drugs is effective against gut and breast tumors including a broad range of others. They are limited by gut toxicity; and are considered an excellent candidate for this protocol.

Another non-limiting but related strategy for modifying DNA involves the boron modified thymidine analogue, 5-(dihydroxyboryl)-2′-deoxyuridine. This molecule is incorporated into DNA as is BRDU and EDU, if perhaps somewhat less efficiently. It is nontoxic in general. However, because it delivers boron to the target cells, it renders them susceptible to killing by low energy neutron therapy (boron neutron capture therapy). Boron has the exceptional ability to trap low energy neutrons and undergo a resulting chemical transformation. It delivers toxic amounts of energy to the cell, but only to that cell, as the energy is highly localized within a tissue. Thus, cells containing boron are highly sensitive to killing under low energy neutron bombardment. Bombardment with low energy neutrons has relatively little effect upon cells without boron. Recent studies have identified modifications of 5-(dihydroxyboryl)-2′-deoxyuridine in which a complex of borons are added; and as such greatly increase the sensitivity of cells containing it to neutron therapy (carborane structures).

In other embodiments, modifications of simple dideoxythymidine will be considered as molecules capable of being efficiently incorporated into DNA. Deoxynucleotides contain a hydroxyl on the 3′ carbon of ribose. This —OH group then is involved in the ribose-phosphate backbone of DNA. The lack of a hydroxyl on the 2′ position of ribose distinguishes it from RNA and stabilizes DNA. Because of their lack of the 3′ hydroxyl on ribose, these molecules will block DNA chain elongation, and be rapidly cleared from circulation.

Appropriate drugs may mimic the dideoxythymidine chemistry but have a structure the cell would recognize as normal thymidine. It is proposed that alterations at position 2 and 3 of ribose in thymidine be targeted for modification. These include thymidine analogues in which the 3′ —OH group is replaced by another group, one which would be unable to participate in DNA chain elongation but would be recognized as normal thymidine by DNA synthase. Possible reagents involve replacement of the 3′-OH group with a methyl (—CH3), an ethyl (—CH2-CH3), an amine (—NH2), a sulfhydryl (—SH), fluorine at position 3 (—F), or any one of a number of similar groups. These may include but would not be limited to such groups as, (—C═CH2), (—CH2-CH2-OH), (—CH═CH—OH), (—Br), (—Se). Two such groups could be attached to carbon #3 of ribose. The structure of ribose itself might be altered, such as introducing a carbon in place of the normal ring oxygen, the insertion of a double bond (—C═C—) into ribose. It might also be possible to utilize a thymidine molecule in which the 3′ hydroxyl has been moved to the ‘2 position of ribose.

Another class of drugs potentially suitable in this protocol would have the stereo chemistry of the “3’ hydroxyl altered (i.e., the thymidine analogue of ara-C, Cytarabine; or ara-A, vidarabine; two drugs that have already been used in cancer therapy and anti-viral studies).

A second strategy that might be considered is to include an alteration of the thymidine that eventually would render it unstable or reactive. This would allow the incorporation of the analogue into DNA which would then lead over time to crosslinking of the DNA, or breakage of the chain. A thymidine analogue that might inhibit DNA polymerase would also be useful. Recall that in BRDU and EDU the methyl group in thymidine (that distinguishes it from uridine) was replaced with Br (BRDU) or by two nitrogens triple bonded to one another (EDU). Replacement of the methyl with any one of the groups listed above might create an unstable DNA product. Central to this chemotherapeutic strategy is the identification of a drug that will work properly in it as described above. An aspect of this application is the use of a unique strategy to demonstrate the utility of a drug in this specific chemotherapeutic strategy. The strategy and overall principles involved in drug identification are an aspect of this disclosure.

The strategy utilized to identify appropriate drugs has two priorities. First, the drug must be specifically toxic only to cells that are replicating DNA, S phase cells. Second, the drug must be rapidly cleared from circulation. These attributes are founded upon the overall chemotherapeutic approach and are central to it. The drug development goals are considered to be protected aspects of this disclosure.

While there may be a variety of means by which a drug can be identified to meet the requirements listed above, a non-limiting example of a strategy is outlined below. It begins in cell culture to identify candidates. Candidate drugs will be tested in animals following IACUC approval. These drugs will then be tested for their effectiveness in killing animal tumors. Priority will be given to naturally occurring animal tumors, but testing may not be limited to them.

A successful drug must be specifically toxic only to cells actively replicating DNA. Toxicity to cells in other cell cycle phases, or resting cells, must be extremely low. A nucleotide analogue like BRDU or EDU would be appropriate. Extensive labelling studies have shown that they are only incorporated into DNA during S phase. Unlike BRDU and EDU, however, a successful drug must kill the cell following incorporation into DNA.

The drug must be rapidly removed from circulation. This is required since it must be toxic only to cells currently in S phase, not to cells that enter S phase after treatment. Again, this is characteristic of both BRDU and EDU.

Tritiated thymidine is an outstanding reagent for killing only S phase cells in culture. Once incorporated its radioactivity kills the cell. Because cells in culture are generally spaced some distance apart (particularly fibroblasts), and because the energy of tritium is extremely low, neighboring cells are not killed efficiently. This reagent would most likely not be appropriate for a chemotherapeutic drug in humans, because of its radioactive nature, and because the tritium would most likely kill neighboring cells tightly packed together in tissues. Tritiated thymidine in culture, along with BRDU and EDU will be used to identify potentially useful drugs as follows:

IDENTIFICATION OF A CANDIDATE DRUG IN CELL CULTURE: Fibroblasts grown in culture (NIH3T3 cells for example), will be treated with tritiated thymidine for 20 minutes, and the thymidine removed. The cells will then be pulsed with BRDU which will similarly be removed after 20 minutes. The cells will then be cultured various times, approximating the cell cycle length of 18 hours, and pulsed with EDU. After another 10 hours to allow all treated cells to die, the culture will be fixed, stained for BRDU and EDU, and analyzed.

The first experiment with tritiated thymidine will allow determination of appropriate analytical conditions, such as timing between different treatments. It will also indicate the profile of a successful drug. While tritiated thymidine will not be a suitable drug to treat human cancer, it will mimic such a drug. The study of tritiated thymidine described above, therefore, will direct establishment of a procedure to identify other appropriate drugs. The procedure described above will be modified based upon results with tritiated thymidine to establish the best protocol to be used in drug identification.

ANTICIPATED RESULTS of studies in cultured cells: Fibroblasts in culture have widely varying cell cycle lengths, so the results will not be expected to be as clean as in tissues, where cells grow with similar cell cycle lengths. However, there is sufficient synchrony in cultured cells to allow the preliminary identification of appropriate drugs.

Tritiated thymidine will serve as a positive control. The procedure described above will be performed and the cells analyzed. It is anticipated that tritiated thymidine treated cultured cells will have minimal labelling with BRDU, because it was added soon after thymidine, and therefore the cells able to incorporate BRDU would also have incorporated thymidine and would die. On the other hand, the cells labelled with EDU hours later would be in a new S phase at the time of addition, and therefore unaffected by thymidine and therefore viable. The actual analyses will, therefore, be determined by experimental analysis of the ratio of EDU to BRDU labelled cells. The ratio of EDU to BRDU should be high and would indicate efficient killing of S phase cells by thymidine. Other test drugs will be judged against thymidine. The higher the EDU/BRDU ratio, the more effective the drug.

Once the results with tritiated thymidine are in hand, the other candidate drugs discussed above will be tested. These are drugs that have been extensively studied in past anti-viral and anti-cancer studies. Some may actually be useful in other anti-cancer strategies. If the above-described drugs are unsuitable, screening of a library of small chemical to find lead compounds with the appropriate activity may be performed.

The experiments described above will be conducted in a 96 well plate containing NIH3T3 cells at 25% confluence. The drug at varying concentrations will be added to specific wells, washed off, and the cells treated first with BRDU and then with EDU as described above. Finally, the cells will be stained with antibody against BRDU and with EDU. The ratios will be determined first by observation, and then by fluorescence photography followed by statistical analysis.

VALIDATION IN ANIMALS: When a suitable drug or group of drugs is identified in culture, approval to use these drugs in animal studies will be obtained from the IACUC. Following approval, an experimental protocol similar to that in culture will be conducted, except that the drugs will be allowed to be cleared by from the blood stream naturally. Thus, the drug will be injected into animals, followed 30 minutes later by an injection of BRDU and several hours thereafter by EDU. The staining of gut crypt cells will be analyzed in paraffin sections. A suitable drug will have a high EDU/BRDU ratio, as in culture.

To complement the studies of gut crypt cells, spleen and bone marrow cells will be collected, stained, and analyzed by FACS analysis or in smears by fluorescence microscopy. These analyses will indicate the general utility of a particular drug.

VALIDATION OF OVERALL APPRAOCH: Following the identification and validation of a candidate drug by studies in mice, it will be possible to test the drug in the actual chemotherapeutic protocol to determine its ability to block the growth of tumors as opposed to normal cells. Several considerations have already been discussed and will be summarized here.

MIN mice, which lack the APC gene and produce spontaneous gut tumors at 4-5 months of age, will be utilized. Other naturally occurring animal tumors and potentially transplanted tumors will also potentially be tested as follows. Once the tumors have formed, the drug(s) identified above at the concentrations known to be just less than toxic levels will be given in multiple doses. These doses will be separated by the appropriate cell cycle interval (approximately 12-14 hours). These treatments will be repeated as often as the animals tolerate them over a period of one month. The number and timing of injections will be determined by toxicity studies. The effect of these treatments upon the survival and growth or tumors will be determined by histological analyses followed by statistical analyses. Efforts will be made to improve the effectiveness by moderation of the dose, frequency, and timing of drug administration. An alternative procedure will involve treatment with the above protocol prior to formation of naturally occurring tumors to determine if tumor development can be blocked.

The treatment methods of the present disclosure may further include determining the cell cycle length of the cell type(s) of interest (e.g., gut and/or lymphoid cells) prior to the administration of the first dose. This may be useful where there is individual-to-individual variance in the average cell cycle length and the individualized treatment may be designed for a particular patient. This step may include staining biopsied tissue(s).

For this procedure to be applicable to humans, cell cycle lengths can be determined. Injection of BRDU and EDU, used in mice, may not be appropriate. Rather the length of the cell cycle can be determined in tissues with the use of three different antibodies that detect three critical markers of cell cycle position. Antibodies against cyclin D1 stains cells in G1 and G2 phase. Antibodies against cyclin E stain cells in S phase. Antibodies against PCNA stain all cells that are actively growing or are likely to be actively growing in the near future. These antibodies can be used to stain biopsied human tissues, and together indicate the cell cycle characteristics of these tissues.

An example of how the three antibody stains described above might be used to calculate cell cycle time is described here as an example. The use of these antibodies in cell cycle length determination, however, will not be limited to the considerations described below, which are just an example. The ratios of cells stained with cyclin E (S phase cells) to cells stained with cyclin D1 (G1 and G2 cells; but only in solid tissues) would give an estimate of cell cycle time. This is because S phase is believed to be rather consistent, and alterations will most likely be seen in G1 phase. This is because it is believed that G1, G2 and S phase have minimal required lengths. When these minimal lengths are added together, they equal about 14 hours for tested cells in mice. Thus, the shortest cell cycle length normally possible is 14 hours. It is further believed that for the cell cycle length to increase, it would be due to an increase in the length of G1 phase. These lengthened G1 cells would likely not contain cyclin D1 and would be stained with PCNA alone. Therefore, the proportion of cells stained only with PCNA would indicate the proportion of cells with cell cycle length in excess of the minimum. The proportion of these cells would indicate the increase in cell cycle length over the minimum. Therefore, by careful analysis of cells in a human tissue with these three antibodies, an indication of the cell cycle characteristics of the tissue can be determined.

In practice a small amount of gut tissue could be obtained, perhaps by a needle biopsy. This tissue could then be stained simultaneously by these three antibodies, and the proportion of cells stained by each determined using quantitative image analysis techniques. There would be three groups of cells thus identified. Those containing PCNA and cyclin D1; those containing PCNA and cyclin E and those containing only PCNA. As described above, a comparison of these three proportions should make it possible to estimate the cell cycle length of the tissue being studied.

Once a general suggestion regarding the length of gut cell cycle length is determined by staining, the next step in designing a treatment strategy for humans would be to administer the treatment drug at various times (for example 1-4 hours longer than the cell cycle time, or 1-4 hours shorter than the cell cycle time). The relative toxicity of these treatments would define precisely the timing best tolerated by the patient, and thereby the length of the cell cycle of susceptible normal cells. This approach would be designed such that those treatment times that are not optimal would induce only slightly noticeable discomfort to the patient; sufficient only to allow the patient to determine the optimal timing. Alternatively, blood analyses might be performed to assess the effect of a treatment upon lymphocytes. Such a treatment may involve a drug concentration below the threshold able to produce noticeable consequences in the patient.

To increase the effectiveness of this treatment strategy, it might be beneficial to perform preparatory treatments to increase normal cell cycle synchrony; and thereby render the patient more likely to have a positive outcome. For example, it might be possible to administer, over a time period (e.g., 2-14 days), subclinical doses of the drug able to kill specifically S phase cells. The treatment strategy could be identical to that described for cancer treatment, except that the doses of drug could be reduced such that no noticeable effects would be felt by the patient.

In practice, the drug could be administered repeatedly at the length of the normal cell cycle. Cells that do not fall within this average cell cycle length would be killed over time. After these cells are eliminated, they would most likely be replaced by normal cells with a more typical cell cycle length. Thus, when the level of drug is increased to toxic levels able to kill the tumor, the normal cells will be in a more uniform state, and therefore more likely to be protected by proper spacing of drug treatments as described here. It is assumed that it would be much more likely to induce a greater degree of synchrony in normal cells, with a naturally high degree of synchrony, than in tumor cells which are essentially asynchronous.

While in normal cells the length of S phase is rather short (e.g., less than 6 hours), it may be much longer in some tumor cells. This means that it might be possible to give two treatments of anti-S phase drugs and have the combined effect observed in a single S phase, but only in the tumor cells with the longer S phase. For example, it might be possible to space two drug treatments apart by 6 hours. If these treatments were spaced exactly 6 hours apart (or a timing based upon determined S phase length), normal cells would incorporate only one dose of the drug into their DNA. On the other hand, a proportion of tumor cells, with longer S phase, would receive a double dose, and therefore be more likely to be killed. Of course, only a proportion of the tumor cells would be positioned in the cell cycle to receive this double dose, but it might be possible to design the dose so that only a double dose would be toxic. Thus, while only a proportion of tumor cells would be killed, normal cells in general would be spared.

The following examples are provided to illustrate the devices and methods of the present disclosure. The examples are merely illustrative and are not intended to limit the disclosure to the materials, conditions, or process parameters set forth therein.

### EXAMPLES

Cell Cycle Length of Crypt Cells

The cell cycle length of crypt cells of mouse gut has been determined with the use of BRDU and EDU injections. These markers are both incorporated into DNA, but only in cells in S phase of the cell cycle. The injection of BRDU was followed at various times thereafter by a second injection of EDU. This approach not only identified the length of the cell cycle in crypt cells but lead the astonishing observation that the length of the cell cycle in crypt cells is surprisingly uniform. Subsequent studies have demonstrated the same fact for spleen lymphocytes. Naturally occurring tumor cells, on the other hand, do not have such uniformity in cell cycle length. These observations lead directly and unavoidably to the conclusion that the uniformity of cell cycle length in normal cells may be a means to distinguish them from tumor cells in a novel chemotherapeutic approach.

The Strategy Utilized to Determine Cell Cycle Length:

It has been estimated in the literature that the cell cycle would be between 12 hr and 2 days in the crypt cells of the gut. These are the cells that are constantly dividing and give rise to mature gut cells forming the lining of the intestinal tract. In order to conclusively demonstrate the length of the cell cycle in mouse gut crypt cells, two separate means of labeling S phase cells in living animals were utilized. Both EDU and BRDU are incorporated only into DNA of cells actively synthesizing DNA, S phase cells. Both are rapidly removed from circulation if not incorporated into DNA, so that a cell containing EDU or BRDU in its DNA would have been in S phase at the time of injection, or shortly thereafter. Indeed, an injection of either marker is effectively a 1 hr pulse with that marker. BRDU is identified with an antibody stain, while EDU is identified by a separate chemical stain. Different fluorochromes are used to identify the two stains, so that cells which incorporated either, or both, can readily be identified. With the use of our image analysis approach, the labeling with each fluorochrome can be accurately quantitated.

Thus, with EDU and BRDU, it was possible to determine which cells had been in S phase at two different times. BRDU was normally injected intraperitoneally (IP) into animals at time zero. At varying times thereafter, EDU was injected IP (although the results did not vary when EDU was injected first). 40 minutes following the EDU injection the animals were euthanized and tissues collected, imbedded in paraffin, and stained. It was possible to tell which cells had been in S phase at the beginning of the experiment from the BRDU stain. Cells that had previously been in S phase could then be compared to those cells that were in S phase at the termination of the experiment from the EDU stain. Therefore, by comparing the BRDU and the EDU stains, it was possible to monitor progression from one S phase to the next as a function of time between the injections. This information, in turn, would directly indicate the length of the entire cell cycle.

Quantitative Image Analysis Strategy:

All analyses were performed by quantitative image analysis. In this procedure tissues are fixed, imbedded in paraffin, section and stained for BRDU, EDU and DNA. Each stain utilizes different fluorochromes so that it is possible to distinguish them by the color of fluorescence. Images of each fluorochrome can then be taken using a sensitive CCD camera of identical regions of the tissue. Most commonly, however, images were taken with a confocal microscope. In the resulting DAPI (4′,6-diamidino-2-phenylindole) image, an outline of each cell was drawn to identify the region of the image occupied by an individual cell. This outline was then overlayed over all separate images to quantitate the fluorescence intensity associated with each stain. The Metamorph image analysis program was then used to determine quantitatively the intensity of each stain in each individual cell. Because of the analytical power of the CCD camera and fluorescence labelling, the levels of each fluorochrome can be determined with great accuracy and compared graphically.

Experimental Methods:

Mice were injected IP with BRDU (2 mg) and then with EDU (2 mg). The injections were separated by from 0 hr to 23 hr. 40 Min following the EDU injection tissues were collected, fixed in 10% formalin, imbedded in paraffin, sectioned (4 microns), stained with an antibody against BRDU, with DAPI and for the EDU with the click-it chemical stain. The stained tissues were imaged in a confocal microscope (40×) such that individual images of each fluorochrome were simultaneously collected with different lasers and filters. The resulting images were then analyzed to quantitate the fluorescence intensity of each fluorochrome in each cell. For this purpose, the DAPI image was utilized to identify individual cells. These were outlined by hand in the Metamorph program, which then was able to identify the region occupied by each individual stained cell. This region was used to quantitate each of the fluorescence images. The results were plotted with the average EDU intensity vs. the average BRDU intensity for each cell. EDU was plotted on the y-axis, and the BRDU on the x-axis. Clearly, cells along either axis had been labelled only by the corresponding fluorochrome with little or no staining by the other fluorochrome. On the other hand, when a cell is located in the center portion of the graph, it clearly had been labelled with both EDU and BRDU.

Results:

Dual labeling with BRDU and EDU was observed when the two injections were closely spaced. For example, if BRDU were injected at the beginning of S phase, and EDU at the end of S phase, the cell would be double labelled. In these experiments, such overlap was observed when the two injections were separated by 0-3 hr and reduced to low levels at 4-6 hr. When the two injections were separated by 7-10 hr, there were few if any cells labelled with both fluorochromes. This indicated that the cells labeled with BRDU had exited S phase prior to the EDU injection. Sufficient time had not transpired for the BRDU labeled cell to re-enter a second S phase and also become labeled with EDU.

An important observation, however, was the reappearance of overlap when the two injections were separated by 12-16 hr. In this case, the cells in S phase at the time of BRDU injection had clearly left S phase, passed through the entire cell cycle, and re-entered S phase of the subsequent cell cycle. In these experiments, the greatest secondary overlap was observed when 14 hr separate the two injections, indicating a cell cycle length of approximately 14 hr.

This type of analysis was repeated three times. The results indicated each time that greatest dual labeling is observed when the two injections are spaced 14 hr apart. Moreover, it was clear that there is a distinct peak in labeling at 14 hr in each experiment, and that the extent of dual labeling in these cells is quite high (up to 60%). This indicates a high degree of uniformity of the cell cycle length in these cells. A much broader peak, and a lower overall total number of dual labeled cells would be expected if there were a great diversity in the overall cell cycle length between different cells of the crypt tissue.

Conclusions from Cell Cycle Length Analyses:

There are several conclusions from the dual labeling experiments described above. First, it is clear that the average cell cycle length in these cells is approximately 14 hr. This result supports the shortest estimates reported in the literature. In addition, it is clear that S phase is less than 5-6 hr in length. This is the point at which double labeling disappears. Mitosis is generally about 1 hr in length. Thus, the combined length of G1, G2 in these cells is approximately 7 hr. A more surprising result, however, is how sharp the dual labeling peak at 13-16 hr. appeared. It is known that many BRDU labeled cells migrate out of the crypt area and cease dividing. These cells, however, retain high levels of BRDU staining. Approximately 14 hr is required for a BRDU-labeled cells to leave the crypt and stop dividing. Therefore, a significant proportion of all BRDU labeled cells would have stopped dividing at the longer times of this experiment. They would never be double labeled. Thus, when a dual labeling percentage over 50% and extending over a 3 hr period is observed, it is apparent that there is a high degree of similarity in the cell cycle length of individual crypt cells. Recall that efficient dual labeling is observed only for 5 hr at the beginning of the experiment due to the short length of S phase. A similar length of time is observed for the overlap during the second S phase.

Chemotherapeutic Predictions from Crypt Cells:

The observation that crypt cells exhibit a high degree of uniformity in their cell cycle lengths length is both surprising and informative. In culture, no such similarity of cell cycle length is observed. Until proven otherwise, observations made in culture are almost always assumed to apply in living tissues. Therefore, until these experiments were performed, it was assumed that there would be little uniformity in normal cell cycle length. If normal cells have a relatively uniform cell cycle length, this might be a means to distinguish them from tumor cells, which are almost certainly heterogeneous in character and therefore cell cycle length. Chemotherapy depends upon distinction between normal and tumor cells. If it can be shown that there is a difference in their cell cycle length uniformity, this would be a powerful tool to be used in chemotherapeutic distinction. Since gut toxicity is one of the most important dose-limiting factors in chemotherapy, a means to reduce gut toxicity would be of great utility in a chemotherapeutic treatment.

Spleen Cells:

Chemotherapy is limited by toxicity to normal cells. Crypt cells are commonly affected by chemotherapy, and therefore often limit the doses, and thereby the effectiveness of tumor killing. The fact that these studies were performed on cells which are commonly most adversely affected by anti-tumor treatments, is significant. However, toxicity to the immune system is also often limiting in cancer therapy. Immune cells are formed initially in the bone marrow, enter the blood, and are retained in various organs. The spleen is important in the storage and handling of immune cells. It was therefore important to determine the cell cycle characteristics also of dividing cells in the spleen. Proliferating cells in the spleen were studied exactly as described above for the crypt. Initial studies involved fixing the whole spleen, imbedding in paraffin, sectioning, staining, and image analysis. Subsequent studies involved releasing immune cells from the spleen and staining them in suspension in preparation for analysis in FACS analysis.

The results with spleen cells were highly similar to crypt cells. There was an initial period of dual labeling with BRDU and EDU that lasted no longer than 5 hr, as seen in the crypt. There was then a period with a limited number of double labeled cells. As with crypt cells, the proportion of double labeled cells increased again. This time, there was an increase clearly seen beginning at 10 hr, and terminating at 16 hr; with the maximal overlap at 12-14 hrs. Thus, the period of maximal dual labeling, which indicates the length of the cell cycle in spleen cells, was slightly shorter than for crypt cells, although the maximal overlap was quite similar. Moreover, the second peak of dual labeling involved over 50% of all cells. As with crypt cells, this indicates a high degree of similarity between the cell cycle lengths of different spleen cells. It is concluded that the length of the cell cycle for spleen cells is close enough to those of crypt cells that an experimental tumor treatment strategy could well be designed to protect both cell types.

The observation that spleen cells, like gut crypt cells, display a high degree of uniformity in cell cycle length demonstrates that it might be possible to also protect lymphoid cells with a cell cycle based chemotherapeutic approach. Because of the similarity of cell cycle length, it might even be possible to protect both lymphoid and gut crypt cells at the same time. However, such a conclusion depends upon what cells are labeled in the spleen studies. For this purpose, two monoclonal antibodies were obtained to detect both mature T cells and B cells.

Identity of the labeled spleen cells: The goal of these studies was to determine if the cells labeled with BRDU and EDU in the spleen actually mature into B cells and T cells. If so, protecting them in a chemotherapeutic protocol would protect the formation of mature lymphocytes. To this end, mice were injected with EDU either 1 hr or 18 hr prior to collection of spleen cells. The cells to be studied were mechanically flushed from the spleens of these animals, and therefore represent blood cells in temporary residence in the spleen. The collected cells were treated with a mild detergent to permeabilize the cells without disturbing their cell surface markers. This detergent (1% saponin) also removed red blood cells. Cells were stained for EDU to detect cells in S phase; and also with a mixture of both CD3 and CD45 antibodies to detect either B cells, or T cells. In spleen cells pulsed with EDU 1 hr prior to collection of the cells, it was found that approximately one third of the cells labeled with EDU contain either B cell or T cell markers. 18% of these cells actually contain a high level of these lymphocyte markers.

This rather low number of marker-containing cells in the spleen is not surprising given the fact that CD3 and CD45 antibodies detect markers displayed most prominently on mature cells. Cycling lymphocytes would be expected to express reduced levels of these markers. However, after 18 hr following the administration of EDU, the proportion of EDU labeled cells with lymphocyte markers increases to over half, with 29% containing a high level of B or T cell markers. These observations suggest that a large proportion of the cells in S phase studied above in the spleen will eventually mature into B cells, or T cells. The preservation of these cells during chemotherapy would be expected to provide at least some degree of protection against immune suppression.

Studies with bone marrow cells are ongoing. Further study will address individual lineages within the bone marrow to determine their cell cycle characteristics. For the purposes of this application, however, the following facts are established:

General Conclusions from cell cycle studies:

1—Both proliferating gut cells of the crypt, and proliferating spleen cells divide rapidly and with a high degree of uniformity of cell cycle length.

2—While the spleen cells have a slightly shorter cell cycle length than the crypt cells, there is enough overlap in their labeling profile that it should be possible to select a strategy to protect both using the cell cycle-length pulse strategy described below.

3—Based upon these observations, the cell cycle-based chemotherapeutic approach described here has the potential to improve current chemotherapeutic approaches. Even if it only achieves a reduction in patient discomfort, it would be of importance. However, the potential that this approach might provide a means to more specifically target tumor cells is of profound importance. The fact that this approach utilizes a fundamental, biological difference between normal and tumor cells makes this a real possibility.

Protective Chemotherapy Based Upon Normal Cell Cycle Length

The results described above were unexpected in the degree of uniformity of cell cycle length observed in the normal tissues tested. These tissues are those most commonly adversely affected by typical chemotherapeutic approaches, and which therefore limit the effectiveness of chemotherapy. Moreover, it should be remembered that traditional chemotherapeutic approaches involve partial toxicity because of the need to spare normal cells. Partial killing strategies promote the formation of tumor cells that are resistant to the drugs involved. The goal of the following approach is to protect normal cells while using a highly toxic dose of the proper toxic drug to avoid drug resistance.

Tumor Cell Studies:

Throughout the above discussion, focus has been placed upon the uniformity of cell cycle length in normal crypt and spleen cells. For a treatment approach to spare normal cells at the expense of tumor cells, it is necessary that similar uniformity in cell cycle length is not present in tumors. On the surface, this conclusion seems obvious since tumor cells have lost the normal control characteristics and growth parameters characteristic of normal cells. It was, nevertheless, essential to this application that this fact be conclusively demonstrated. For this demonstration, the Min mouse tumor model was selected. It is known to produce naturally occurring tumors of the gut. Min mice have one copy of the APC gene deleted. This gene is considered a gate keeper gene in the formation of gut tumors. The absence of one copy of this gene leads within 4-6 months to the formation of numerous gut tumors (after the spontaneous loss of the only remaining APC gene). They have the characteristics of naturally occurring gut tumors and are considered among the best possible models for the formation of natural tumors. To emphasize this point, loss of the APC gene is considered an important step in the formation of many human gut tumors. Therefore, the Min mice and normal human gut tumors have a very similar molecular basis.

A Min mouse colony (founders obtained from Jackson Labs, Me.) was established in the Cleveland State University Animal Research Facility according to a protocol approved by the Cleveland State IACUC. Mice were genotyped with PCR and marked with an ear tag. Positive mice are heterozygous (Min+/−) and are crossed with normal BalbC6 mice. Positive Min mice are identified as having both one normal and one mutant copy of the gene. After approximately 5 months of age, and before 6 months of age (or any indication the animals are under stress) the experiments described below were performed. The tumors were collected, imbedded in paraffin, sectioned, and stained exactly as described above for normal tissues. BRDU was injected at 0 hr and stained red. EDU was injected just prior to tissue collection at the indicated times and stained green. DNA was stained blue with DAPI. Actively growing cells were stained yellow with PCNA. These four colors were imaged with confocal photography.

The goal of these studies is to determine if there is a difference in the cell cycle length of normal vs. tumor tissues. Before a quantitative analysis of the cell cycle length in tumors was initiated, however, it is important to understand the variability in morphology of different tumors. Recall that this mouse model system was chosen because it mimics the formation of normal human tumors. In many human gut tumors, the loss of an APC gene is one of the early steps in tumor formation. Such human tumors would be expected to be formed and to behave very similarly to these Min tumors. Accordingly, the appearance of individual tumors varied as would be expected for the variety of naturally accruing gut tumors in humans. Some tumors had morphological features quite similar to normal cells, while others bore no apparent resemblance to the parent cells. It was anticipated, therefore, that the results of cell cycle length studies in tumors would yield varying results.

A series of three extensive experiments were performed upon Min mice at 5 months of age to determine the cell cycle length of tumor tissues. The experiments were exactly as described above with normal tissues. A separate tumor-bearing animal was injected for each time point, and multiple tumors from each animal were analyzed. Tumors from the small intestine, one inch from the stomach were analyzed, as well as tumors throughout the mid-gut region. The data from all tumors from a given animal are combined. Double labeling in all tumors was observed for the first 4 hr as with normal tissues. This double labeling was reduced when the BRDU injection preceded the EDU injection by 7 hr and 10 hr. This is to be expected, as cells had passed out of the first S phase and did not have time to enter the second. However, with these tumor cells, there was little double labeling at 12 hr. This is important, as considerable double labeling was consistently seen in normal tissues when BRDU injection preceded EDU by 12 hr. Clearly, the cell cycle length in the tumor cells had been delayed compared to normal cells. However, at 14 hr a small amount of double label was observed in some tumors, which increased at 16 hr. (25%-30% of cells). The double labeling decreased at 18 hr.

Several conclusions can be reached from these data. First, it is clear that normal cells divide in general more rapidly than tumor cells, since there is little overlap in BRDU and EDU labels when the two are injected 12 hr apart in tumors, while there is consistent overlap in normal cells at this time. On the other hand, there was observable overlap in the tumor cells at 16 hr, as with normal cells. The peak at 16 hr, however, was brief and relatively weak compared to the huge peak of overlap in normal cells.

Tumor cell cycle length: It would appear that while there are no tumor cells with the short cell cycle length of normal cells, there are some tumor cells with a relatively short cell cycle length. However, it further appears that those tumor cells with even this slightly increased cell cycle length represent only a small proportion of all tumor cells. For most tumors, the cell cycle length is much greater than 12 hr or even 16 hr. This result is in keeping with the overall histological variation of tumors in Min mice. There was a proportion of tumor tissues that bore a resemblance to normal tissues, as they are aligned in rows reminiscent of the crypt cells. However, the proportion of such cells was limited. It is assumed that tumor cells with a high degree of similarity to normal cells possess only a slightly longer cell cycle length. Presumable tumor cells that have little resemblance to normal cells have a significantly different cell cycle length.

From the above data, it is clear that the short and relatively uniform cell cycle length of normal cells is a clear distinction from tumor cells. However, the data also raised the possibility that there might be some overlap between the cell cycle length of normal and tumor tissues at 16 hr. If so, this is an important consideration when it comes time to utilize this difference in the design of a chemotherapeutic approach. Another type of experiment was, therefore, designed to determine the extent to which there might be an overlap in the cell cycle length between normal and Min tumors at 15 hr. In this case, mice were given BRDU and then 12, 15 or 18 hr later injected with EDU. From each animal, tumors from the small intestine and from the mid-gut were analyzed, along with normal tissues adjacent to each of these tumors. Comparison was therefore made between adjoining normal and tumor tissues. The degree of overlap in labeling was carefully determined and then compared for each tissue type. Then for comparison, the degree of overlap at 15 hr was arbitrarily set to 100%, and the relative overlap at 12 hr and 18 hr determined and plotted. This was repeated for each tumor and each neighboring normal tissue region.

The data indicated that a significant proportion of all normal cells had a cell cycle length of near 15 hr, since at 12 hr or 18 hr the overlap was dramatically diminished. No such observation was made in the tumor tissues. There was no specific increase in the number of cells with a cell cycle length of 15 hr in the tumor cells. Indeed, there appeared to be no consistent relationship between cell cycle length at 15 hr in the tumor compared to 12 hr or 18 hr. While in normal cells there is a uniformity of cell cycle length near 15 hr, there is no such uniformity in any of the tumors tested. As predicted, and as required for the chemotherapeutic approach described in this proposal, there is a clear difference in the cell cycle lengths of normal and tumor tissues. It should, therefore, be possible to design a chemotherapeutic strategy to utilize this difference in selective killing of tumor cells.

Anti-Tumor Strategy:

An anti-tumor strategy is based upon the fact that normal cells have a similar cell cycle length and will be protected from repeated treatments of an S phase-specific toxin. Tumor cells, on the other hand, with different cell cycle lengths will be susceptible to killing by the toxin. While the selective toxicity of such a treatment may be limited after a single round, the tumor specific toxicity will become more apparent as this treatment strategy is repeated. As the treatments are repeated, normal cells will be in cell cycle phase rendering them resistant to toxicity, whereas tumor cells will not. Tumor cells with even slightly different cell cycle lengths, or cells that are only sporadically dividing will eventually be killed. Therefore, the uniformity of cell cycle length is selected by this treatment as a means to distinguish normal and tumor cells.

Elimination of drug resistance: One of the most compelling aspects of this strategy may not be immediately apparent. There are two major limitations to anti-cancer treatments. The first has been mentioned, susceptibility of normal cells to the treatment. This procedure is designed to spare particularly spleen and gut normal cells, which are commonly limiting in treatments. The second major drawback to chemotherapy involves drug resistance. This results when cells are treated with the drug and some survive. Those that survive are those whose biochemistry makes them least susceptible. Over time, particularly with repeated treatments, these cells will be highly selected and over grow the rest of the tumor. Before long, the tumor itself is drug resistant. This feature is due to the survival of some treated tumor cells and necessitated by the limitations in drug levels due to killing of normal cells. In this strategy, each drug treatment may be completely toxic to S phase cells. No susceptible cells survive, and the development of drug resistance is thereby limited.

A drug appropriate to this treatment strategy generally possesses certain features. First, it must be toxic only to cells in S phase. Second, the drug must be rapidly removed from circulation. This means that any cell in S phase at the time of treatment will be killed, no other cells will be harmed. In addition, the drug will be present only long enough to affect cells that were in S phase at the time of treatment. Cells entering S phase subsequently will be unaffected. It would of course be optimal to select a drug toxic to DNA synthesis by the major replicative polymerases, without affecting minor polymerases involved in DNA repair or RNA synthesis. Simply timing repeated treatments of current drugs to align with the cell cycle length of normal cells may be an option.

The fact that both BRDU and EDU have exactly the characteristics required for a drug in our treatment strategy is encouraging. They both label cells only in S phase, and they remain in circulation a limited amount of time. They are not toxic but can be used as markers in a proof of principle experiment. The drugs predicted to perform best in this treatment strategy would be expected to behave exactly as BRDU and EDU, except that they would eventually lead to cell death. While the incorporated drug must be toxic, it might be valuable for killing to take place over a period of hours to ameliorate the effect of half the normal cells being poisoned at one time.

The design of new drugs may be driven by the strategy, and processes for drug selection are also contemplated herein. A knowledge of the uniformity of normal cell cycle length leads directly both to the strategy proposed, and to the drug to be used in it. Such a drug would be less likely to be of value in a different strategy.

Proof of Principle:

To validate the above strategy an experiment was designed to identify cells killed by treatment with an S phase-specific drugs administered at various times as proposed above. Both BRDU and EDU behave exactly as the proposed drug. It is, therefore, possible to use these drugs to explore the utility of this strategy in general.

The experiment to validate this procedure was designed as follows: Mice were given BRDU in their drinking water at the beginning of the experiments. This constant supply of BRDU would label all proliferative cells over the length of the experiment. 4 hr after BRDU-containing drinking water was given to the animals, two injections of EDU were given. These injections were spaced 5, 8, 11, 14, 17, and 20 hr apart in separate animals. At 40 min following the final injection tissue was collected, fixed, imbedded, stained, and analyzed as described above. The goal was to determine how many cells were labeled with BRDU and NOT WITH EDU. EDU in this experiment represents the toxic treatment. Any cell labelled with EDU would be expected to be killed by the appropriate S phase specific toxin. The surviving cells would, therefore, be identified by BRDU positive, EDU negative cells. As with many proof of principle experiments, this one has a drawback that must be considered. BRDU remains present in cells that differentiate and move up the villi towards the lumen of the gut. Cells on the verge of terminal differentiation would be labelled with BRDU but cease dividing prior to the administration of EDU. This would result in an over-estimate of the number of BRDU+ and EDU− cells (those that survive the treatments), particularly at later times in the analysis. While this might result in an overestimate of surviving cells at later times in the analysis, it is not considered so complicating as to invalidate the results.

Results with the proof of principle experiment are as expected. The protection increased dramatically as the cell cycle length was approached. Thus, the number of BRDU+ and EDU− cells, those proposed to survive treatment, were low until the two injections are spaced apart by the length of the cell cycle. Almost 40% of the BRDU positive cells remained unlabeled with EDU when the two EDU injections were spaced 14 hr apart. This is strong evidence that if the injection had been an S phase toxin, 40% of the cells would have been protected at 14 hr. On the other hand, the levels remain high at 17 hr and did not fall until the two injections of EDU were spaced 20 hr apart. As indicated above, at later times, BRDU labeled cells will move out of the crypt area and become unavailable for EDU labeling. This is an inherent complication of the proof of principle experiment that would lead to the overestimate of protected cells at longer intervals. The data are, therefore, most appropriate at shorter times of the analysis. With this consideration, the above analysis is a strong validation of the overall treatment strategy. However, while the data at 17 hr may be artificially inflated, it is also possible that because of the cell cycle characteristics of these cells, with an S phase of 5 hr, a treatment slightly greater than the cell cycle length might be most appropriate. Such a possibility will be considered as these studies proceed. So far as this application is concerned, it is clear that a treatment strategy based as described herein will be protective to a large proportion of all normal cells.

General Conclusion:

Studies uniquely led to the identification of a cancer treatment strategy based upon the cell cycle length of normal cells. This strategy relies upon the uniformity of cell cycle length first shown by the inventor. The approach has the ability to protect normal cells over an extended period of time, and to greatly reduce the possibility of drug resistance. It should in principle be appropriate for a broad range to tumors. Moreover, this strategy will be of use in the treatment of hyper- or hypo-proliferative disorder of many types, and perhaps other problems related to cell proliferation.

This written description uses examples to describe the disclosure, including the best mode, and also to enable any person skilled in the art to make and use the disclosure. Other examples that occur to those skilled in the art are intended to be within the scope of the present disclosure if they have structural elements that do not differ from the same concept, or if they include equivalent structural elements with insubstantial differences. It will be appreciated that variants of the above-disclosed and other features and functions, or alternatives thereof, may be combined into many other different systems or applications. Various presently unforeseen or unanticipated alternatives, modifications, variations or improvements therein may be subsequently made by those skilled in the art which are also intended to be encompassed by the following claims.

1. A method for selectively killing cells in S phase, the method comprising:
administering a first dose of a drug selectively targeting S phase cells; and
administering an additional dose of the drug after waiting a full normal cell cycle.

2. The method of claim 1, further comprising:
repeating the administration of the additional dose at least one time.

3. The method of claim 1, wherein the full normal cell cycle is a normal human intestine cell cycle or a lymphoid cell cycle.

4. The method of claim 1, wherein the drug selectively targeting S phase cells is selected from the group consisting of bromodeoxyuridine (BRDU) and 5-ethynyl-2′-deoxyuridine (EDU).

5. The method of claim 1, wherein the drug selectively targeting S phase cells is related to a dideoxynucleoside, or a derivative of such a drug specifically designed to enter tissues and be incorporated efficiently into DNA, and then block chain elongation.

6. The method of claim 1, wherein the drug selectively targeting S phase cells is related to a thymidine analogous, or a derivative of such a drug, specifically designed to enter tissues and be incorporated efficiently into DNA, and then render the DNA unstable or toxic within the next few cell cycles.

7. The method of claim 1, wherein the drug selectively targeting S phase cells is selected from the group consisting of methotrexate, pemetrexed, pralatrexate, cytarabine, gemcitabine, clofarabine, fludarabine, mercaptopurine, nelarabine, pentostatin, thioguanine, azacytidine, decitabine, irinotecan, and teniposide.

8. The method of claim 1, wherein drug selectively targeting S phase cells is administered orally.

9. The method of claim 1, wherein drug selectively targeting S phase cells is administered via injection.

10. The method of claim 1, wherein the drug selectively targeting S phase cells is tritiated thymidine.

11. The method of claim 1, wherein the method disproportionately kills cancer cells.

12. A process for treating small intestine cancer comprising in sequence: administering a first dose of a drug selectively targeting S phase cells, wherein the drug kills cancer cells in the S phase and normal small intestine cells in the S phase and wherein the drug does not kill cells that are not in the S phase; waiting a full normal small intestine cell cycle; and administering an additional dose of the drug.

13. The process of claim 12, further comprising: repeating the administration of the additional dose at least one time after waiting at least one more full normal small intestine cell cycle.

14. The process of claim 12, wherein the drug selectively targeting S phase cells is selected from the group consisting of bromodeoxyuridine (BRDU), 5-ethynyl-2′-deoxyuridine (EDU), deoxynucleosides, methotrexate, pemetrexed, pralatrexate, cytarabine, gemcitabine, clofarabine, fludarabine, mercaptopurine, nelarabine, pentostatin, thioguanine, azacytidine, decitabine, irinotecan, and teniposide.

15. The process of claim 12, wherein the administration is oral or via injection.

16. A cancer treatment method comprising: administering a first dose of a drug selectively targeting M phase cells; and administering an additional dose of the drug after waiting a full normal cell cycle.

17. The cancer treatment method of claim 16, wherein the drug is vinblastine.

18. The cancer treatment method of claim 16, wherein the drug is selected from the group consisting of 5-fluorouracil (5-FU), tegafur, capecitabine, 5-fluorouridine 5′-triphosphate (FUTP), 5-fluoro-2′-deoxyuridine 5′-triphosphate (FdUTP), and 5-fluoro-2′-deoxyuridine 5′-monophosphate (FdUMP).

19. The cancer treatment method of claim 16, wherein the drug comprises 5-(dihydroxyboryl)-2′-deoxyuridine.

20. The cancer treatment method of claim 16, wherein the drug comprises a thymidine analogue in which the 3′ —OH group is replaced by a group selected from the group consisting of a methyl (—CH3), an ethyl (—CH2—CH3), an amine (—NH2), a sulfhydryl (—SH), fluorine at position 3 (—F), (—C═CH2), (—CH2—CH2-OH), (—CH═CH—OH), (—Br), and (—Se).
